(19) United States
(12) Patent Application Publication
(10) Pub. No.: US 2008/0249394 A1
(43) Pub. Date: Oct. 9, 2008

(54) METHOD FOR IMPROVED ROTATIONAL ALIGNMENT IN JOINT ARTHROPLASTY

(75) Inventors: Nicholas Giori, Stanford, CA (US); Robert Siston, Dublin, OH (US); Scott Delp, Stanford, CA (US)

Correspondence Address: D. BOMMI BOMMANNAN, 2251 Grant Road, Suite B, LOS ALTOS, CA, 94024, US

(73) Assignee: The Board of Trustees of the Leland Stanford Junior University, Stanford, CA (US)

(21) Appl. No.: 12/062,334

(22) Filed: Apr. 3, 2008

Related U.S. Application Data
(60) Provisional application No. 60/909,911, filed on Apr. 3, 2007.

(51) Int. Cl. A61B 5/00; A61B 17/56

(52) U.S. Cl. 600/407; 606/102

(57) ABSTRACT

A method for improved rotational alignment of the bones in joint surgery is described. The method involves the tracking of the relative motion of a third bone in with respect to the movement of the first and second bone. In one aspect of the invention, the motion of the patella is used to derive the axis of rotation of the femoral and tibial components in total knee arthroplasty, either alone or in combination with other techniques.

### FIELD OF THE INVENTION

The field of the invention relates to a novel technique to determine femoral component rotational alignment using computer navigation in total knee arthroplasty.

### DESCRIPTION OF THE RELATED ART

Total knee arthroplasty (“TKA”) involves the replacement of all the articular surfaces of the knee joint. Success in total knee arthroplasty depends, in part, on the proper alignment of the implants. One aspect of this alignment is the internal/external rotational alignment of the femoral component. It has been shown in a previous study that rotational alignment of the femoral component is particularly problematic (Berger et al., 1998). Malalignment of femoral component rotation can lead to a number of complications. Internal rotation of the femoral component causes a shift into valgus alignment with flexion and also an increase in the “quadriceps” (Q) angle with deleterious effects on patella tracking. Internal rotation of the femoral component also causes differences in the flexion and extension gaps by altering the relative dimensions of the posterior condyles in flexion. Flexion then causes asymmetrical tension across the prosthesis and gapping on the lateral side (Anouchi et al., 1993).

The knee joint is complex in its degrees of freedom, which cannot be described by simple hinged members. The femur and the tibia meet at the knee and are separated by cartilage that acts as a bearing surface. The patella is connected to both the femur and the tibia by various soft tissue structures. These soft tissue structures, in combination with the orientation of the quadriceps tendon and the orientation and attachment of the patellar tendon enable the joint to stay in alignment during normal operation. During total knee arthroplasty, the distal end of the femur, the proximal end of the tibia, and the posterior surface of the patella are machined for acceptance of knee replacement components. FIGS. 1-4 show the methods currently used to resect the distal femur prior to total knee arthroplasty. In all of these figures, the right distal femur is viewed from its distal end. As shown in FIG. 1, resection of the femur involves making anterior and posterior cuts 110 and 120 respectively. The epicondylar axis 150 is shown connecting the lateral (130) and medial (140) epicondyles of the femur. In general, resection is performed by affixing a cutting guide to the femur, tibia and patella, respectively, and utilizing a surgical saw to remove a thin layer of the arthritic bone and cartilage surface of the femur, tibia, and patella. Further machining of the bone, such as drilling holes and cutting slots, is then performed to prepare for placement of the knee replacement components. As is well known, these steps require precision and accuracy if the total knee arthroplasty is to be successful. Proper positioning of the implants requires proper positioning of the bone cuts, holes, and slots. This is achieved using instruments that reference visible external anatomical landmarks or the intramedullary canal of the femur and tibia. The femoral component is generally attached to the end of the femur using a special acrylic bone cement, or by obtaining a tight “press fit” of the femoral component to the femur. The outer surface of the femoral component comprises one of the replacement knee surfaces.

The components also include a tibial component, which fits onto the cut surface of the tibia. The tibial component is generally attached to the upper end of the tibia using bone cement or screws. In modular implants, an ultra high molecular weight polyethylene insert then attaches to the proximal surface of the tibial component.

Optimally, the anterior and posterior cuts on the distal femur are aligned with the epicondylar axis. FIGS. 2 and 3 show rotational error of the anterior and posterior cuts in the internal and external directions, respectively. Various methods are currently used to minimize the rotational error as shown in FIG. 4. One method is to draw the Whiteside's line 410 (Whiteside et al., 1995), which is defined as the line running along the deepest part of the sulcus of the trochlea. The trochlea of the femur is the femoral side of the patella-femoral articulation. Other methods such as projection of the epicondylar axis (150), direct digitization of the epicondyles, and use of the posterior condylar axis (430), have been used for guiding the bone cuts, as illustrated in FIG. 4. It has been reported that accurate localization of the epicondylar axis using these various techniques is difficult even under normal conditions when the joint is not diseased and has not been previously operated on. It is particularly difficult in the diseased or previously operated joint when external bony landmarks are distorted or no longer available. It will be beneficial to have a method that does not possess these drawbacks.

Computer-assisted surgical navigation systems have been developed in an effort to align implants more accurately than is possible with use of traditional mechanical guides. Surgeons using these systems have reported more accurate alignment of implants in the frontal plane. There are three types of computer-assisted navigation systems for total knee arthroplasty. Image-based systems use either intraoperative fluoroscopic images or preoperative computed tomography scans to guide the placement of components. Image-free systems are based on anatomic landmarks that are located intraoperatively through direct identification or kinematic algorithms. Many alignment devices comprise conventional instrumentation (such as an intramedullary alignment rod) and drilling into the bone to anchor the alignment tool in order to ensure accuracy (U.S. Pat. No. 5,445,642; U.S. Pat. No. 6,595,997; and U.S. Pat. No. 7,104,997). This level of intrusion is not desirable, because it increases the risk of fat embolism and unnecessary blood loss in the patient.

Robotic systems use machines that either guide the surgeon or perform cuts during portions of the operation. Such systems combine surgical planning software with a registration method to implement surgical plans for orthopedic procedures. The “Robodoc” hip replacement system from Integrated Surgical Systems (Sacramento, Calif.) uses a computer-based surgical plan with a robotic manipulator to perform intraoperative registration and some of the bone resections needed for hip replacement. The Robodoc system has been tested in the operating room and has produced accurate bone resections, but the system has several important limitations. It is expensive, for example, and must be operated by a specially-trained technician. It also adds substantially to OR time, increasing the cost of using the system. Another problem is that the Robodoc system uses a pin-based registration method, which increases patient trauma and lengthens the patient's rehabilitation time.

The method of Delp et al. (U.S. Pat. No. 5,871,018) consists of acquiring radiologically generated anatomical data on the joint and constructing a 3-dimensional image of the internal structure that would be used to perform the surgery. Carson et al. (U.S. Pat. No. 6,923,817) describe a system and process for total knee arthroplasty that uses tracking of the relative positions of the patella and the femur and digitally storing images of the anatomy using X-ray fluoroscopy. However, the problem of identifying the correct rotational axis is not addressed in these inventions and this aspect is still left to the judgment and skill of the surgeon.

Computer navigation promises to improve alignment of total knee replacement components, but at present, experimental work by several experts shows that the techniques that are used for femoral rotational alignment are no better than traditional techniques that do not use computer navigation technology (Siston et al., 2005). The misalignment of the rotational axis was over 5 degrees in over 82% of the cases where traditional techniques were used. Similarly, in tibial rotational alignment, only 13.1% of the cases had rotational misalignment less than 5 degrees. A computer-aided navigation system that relies on digitization of the epicondyles to establish femoral rotational alignment improved alignment by just 1 degree over a reference traditional method that relied on a surgeon's skill.

As navigation systems become more widely used, it is important to evaluate all aspects of their performance. The results by Siston et al. (2006) suggest that a navigation system that relies on digitization of landmarks to establish a rotational alignment axis does not provide a more reliable means of rotational alignment than using traditional TKA instrumentation. When the tibial tubercle is referenced by the navigation system to establish tibial component rotational alignment, the resultant alignment axes are significantly less reliable than when traditional instrumentation is used. These results contrast the demonstrated ability of navigation systems to improve alignment in the frontal plane. In the frontal plane, the landmarks that serve as the endpoints of alignment axes (e.g., the center of the ankle) may have an error of up to 6 mm and correspond to an alignment error of approximately 1.25 degrees. However, in the transverse plane, an anterior-posterior error of 6 mm corresponds to an error of approximately 4.5 degrees. The presence of osteophytes on the periphery of the knee during a TKA may distort the normal anatomy and the relatively subjective nature of landmarks that are used to determine rotational alignment (e.g., the medial ⅓ of the tibial tubercle) also may contribute to the greater variability in the transverse plane (Siston et al., 2006).

Existing computer based navigational techniques, therefore, do not provide a more reliable means of rotational alignment as compared with traditional techniques. One of the main reasons for difficulties in alignment, even when computer navigation and precision are involved, is the variability in joint geometry and bone shape between individuals that makes a generalized numerical scheme inapplicable. There is, therefore, a need to develop a more accurate and more precise way to set the femoral rotational alignment in total knee arthroplasty that would provide for peculiarities in the individual joint. This is proposed to be solved by deriving the alignment data from the relative movement between the bones at the joint.

### SUMMARY OF THE INVENTION

The invention includes methods and systems to record the alignment axis of two bones meeting at a joint, prior to or during surgery for artificial joint implantation, by using the relative movement of a third bone with reference to the two major bones meeting at the joint. When applied to knee replacement surgery, the alignment axes of the femur, and the tibia at the knee are determined using the movement of the patella. The method can also be used for aligning the femur and tibia using the talus and calcaneus. Similarly, the hip joint can be aligned using the movement of the tibia with respect to the femur. The relative movement of the bones is tracked using either optical sensors, electromagnetic sensors, some other sensor, or medical images. The data on relative movement of the bones can be used as it is or after smoothing using a suitable algorithm. The curve traced by the third bone on the first or the second bone can be fitted to a suitable function such as a spline function or a polynomial function. Optionally, two or more rotational alignments axes, obtained according to two or more different techniques, may be combined to generate a rotational alignment axis that provides improved accuracy in rotational alignment.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a schematic diagram of the distal end of the right femur as seen from distally. It shows the anterior and posterior cuts on the distal femur that are done in preparation for receiving the implant in total knee arthroplasty. The position of the epicondylar axis is shown.

FIG. 2 shows internal rotation of the bone cuts.

FIG. 3 shows external rotation of the bone cuts.

FIG. 4 illustrates the various reference axes used in knee surgery.

FIGS. 5A and B show schematic diagrams of the computer-assisted surgery system for total knee arthroplasty.

FIG. 6 illustrates tracking of the patellar movement across the femur in knee arthroplasty.

FIG. 7 shows how tracking of the patellar movement across the proximal tibia can also define a line for orientation of the tibial component.

FIG. 8 shows rotational alignment errors of several arthroplasty techniques as obtained in a study involving 12 surgeons.

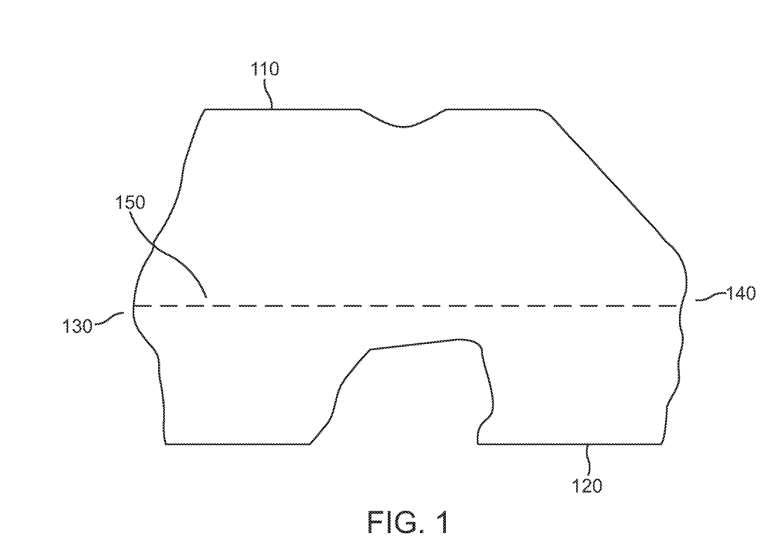

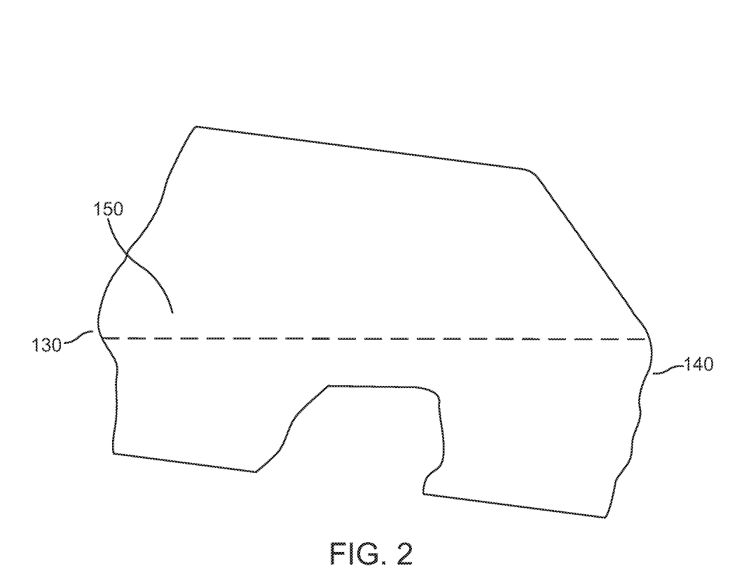

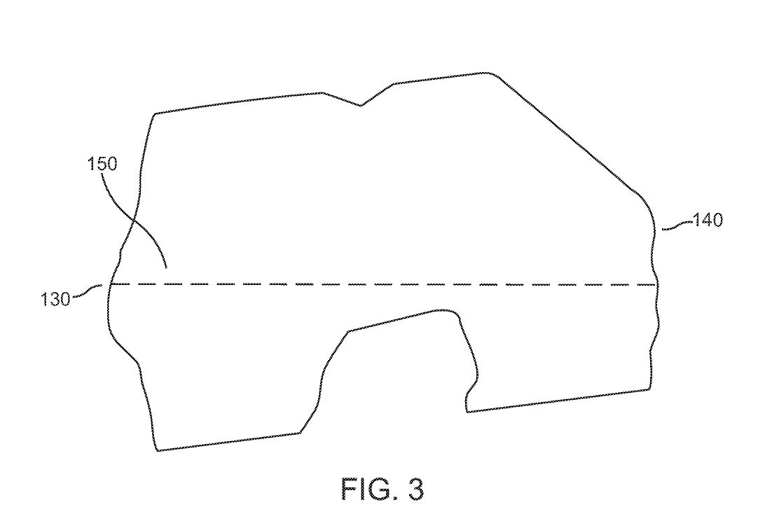

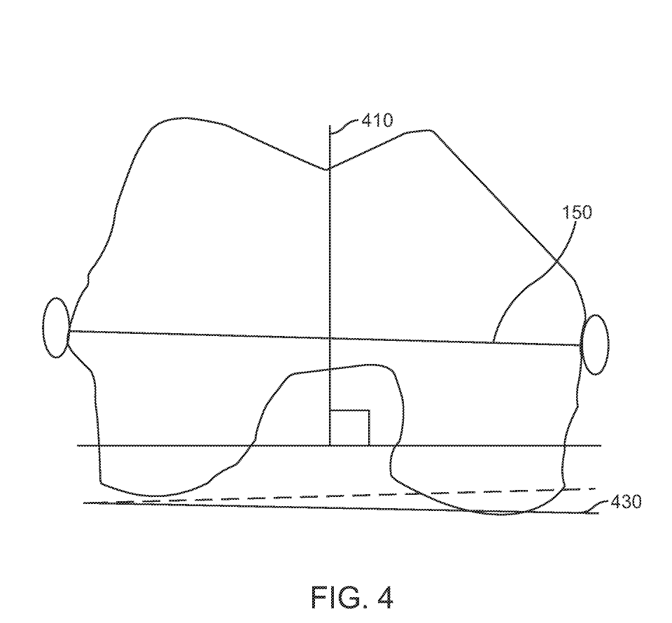

### DETAILED DESCRIPTION OF THE PREFERRED EMBODIMENTS

The invention is a method to record the alignment axis of two bones prior to or during surgery for artificial joint implantation by using the relative movement of a third bone with reference to the two major bones meeting at the joint.

During knee arthroplasty, after the knee has been exposed and trackers have been placed in the femur and the tibia, but before any bone cuts are made, the path that the patella tracks with respect to the femur when the knee is flexed and/or extended is recorded. To do this, the surgeon reduces the patella in the native trochlear groove.

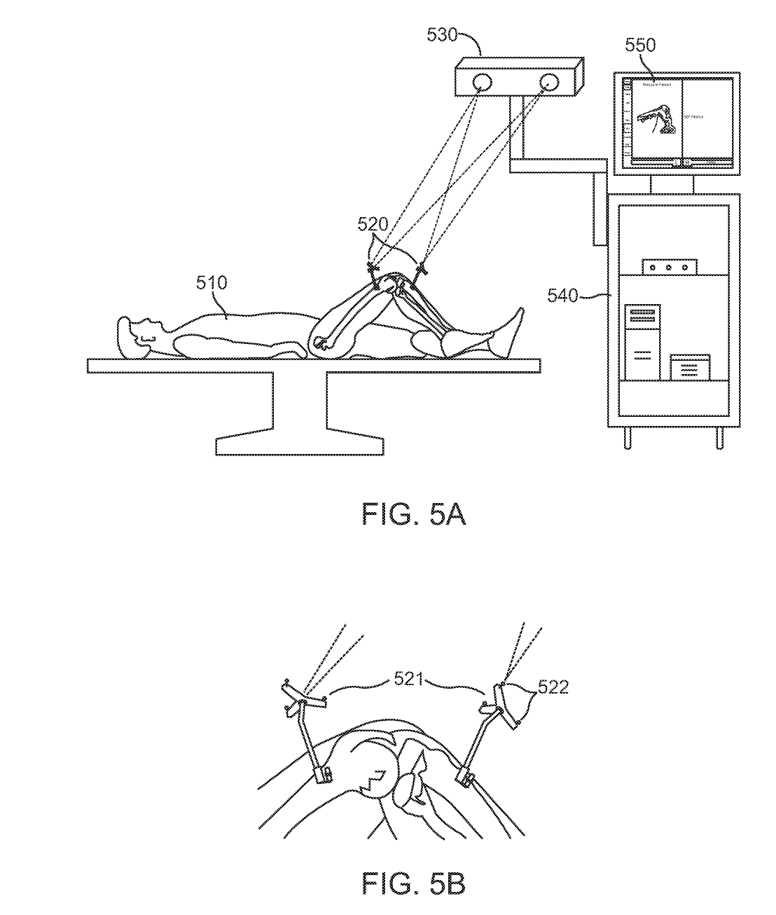

Total knee arthroplasty according to the present invention is shown in FIGS. 5A and B. The right knee of a subject 510 is shown set up for the surgical procedure in FIG. 5A. Reference frames 520 are attached to the femur and the tibia to enable the optical tracking system 530 to reference their respective positions. Details of the reference frames 520 are shown in FIG. 5B, where the three-armed frames 521 with reflective reference indicia 522 are firmly attached to the femur and the tibia. The optical tracking system measures the position and orientation of reference frames that are attached to the patient's femur and tibia and the data are processed and stored by the computer 540. Information such as cut plane orientation and limb alignment are displayed on the computer monitor 550. The surgeon is able to locate the cuts required precisely with reference to the anatomical landmarks with the aid of the image shown on the computer monitor.

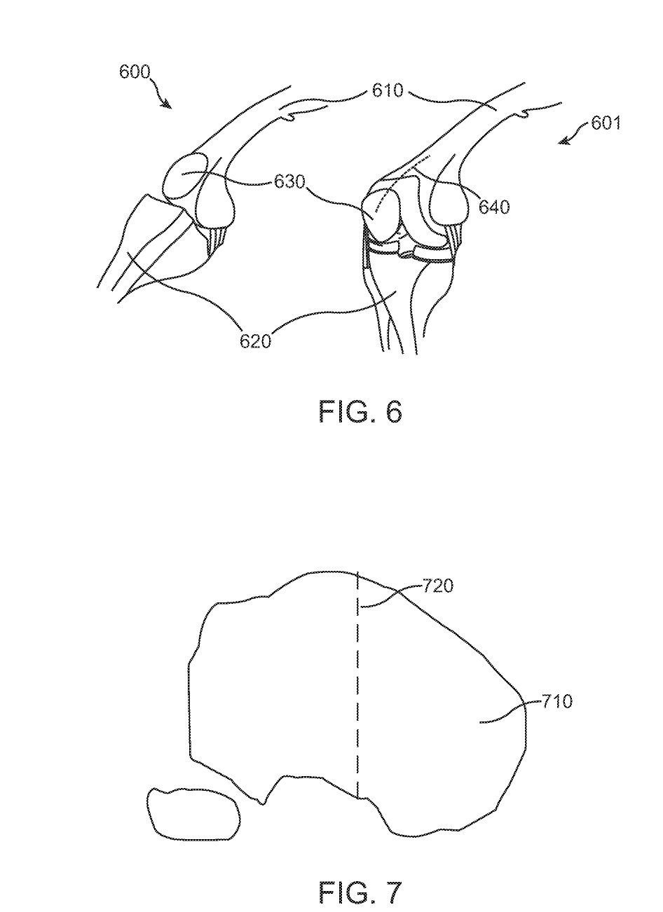

In a preferred embodiment of the invention, the position of the alignment axis on the femoral bone is derived by tracking the position of the patella in the trochlear groove as the knee joint is flexed and/or extended by fixing a computer-tracked point probe to the patella. The position of the point probe and therefore the patella with reference to the femoral groove is tracked by recording on the computer. The tracking procedure is shown schematically in FIG. 6, where the exposed knee joint is shown in the extended (600) and flexed (601) positions. In the extended position, the femur 610 and the tibia 620 are aligned axially and the patella 630 sits on top of the femoral groove. On flexing the knee, the patella moves down in the femoral groove for about 50-60 mm down to the distal face of the femur. The tracking device attached to the patella therefore traces a path 640 as shown in FIG. 6.

The computer system records the path 640 as traced by the tracking device in FIG. 6. Tracking of the patellar movement can be done either before or after exposure of the joint during surgery. The geometrical boundaries of the femur and the tibia are recorded in the computer, numerically and/or as an image. At this stage, the knee is flexed and/or extended and the movement of the tracker attached to the patella is recorded. The tracking line is obtained as a series of discrete positional co-ordinates. For example, using an optical stylus of a navigation system, the surgeon may press a sharp endpoint of the stylus into the anterior surface of the patella and flex and extend the knee joint through a range of motion, such as from approximately 0° to approximately 120°. While the knee is being flexed and/or extended, the navigation system records the position of the endpoint of the stylus with respect to the tracker attached to the femur, thereby recording the three-dimensional path of translation of the patella in the trochlear groove. Alternatively, the tracking may be accomplished through imaging, for example by using X-ray imaging, computed tomography imaging, fluoroscopic imaging, magnetic resonance imaging, or ultrasound imaging.

The data obtained from tracking can either be used in the raw form, or after fitting a suitable functional form such as a polynomial or a spline function. For example, the collection of points may be projected into the transverse plane, and a line may be fit to the collected points that are within an anterior-posterior window of the center of the knee. It would also be advantageous to filter the data using a suitable algorithm. For example, since many problems with patellar tracking are thought to occur between full extension and 20° of knee flexion, an anterior-posterior window of the center of the knee may be used to ensure that only points which represent the patella properly tracking in the trochlear groove are used. In one embodiment, a 30 mm window may be used for this purpose. The tracking line is projected on to the surface of the femur either on the image or numerically. The alignment axis of the femoral component is drawn perpendicular to this line. Once this axis is known, precise cuts to the ends of the femur can be made during the surgical procedure using the computer-assisted surgical tool.

A similar procedure is used for resecting the ends of the tibial component. The position of the tracker on the patella is traced with reference to the tibial contour as shown in FIG. 7. A schematic alignment axis 720 traced by the patellar movement on the tibia 710 is shown in this figure. The alignment data is again processed using numerical means such as noise filtering and curve fitting as with the femoral component. The curve is then projected on to the proximal end of the tibia and the axis of rotational alignment is determined to lie perpendicular to the projected line. After the bone cuts are made, the prostheses are attached using bone cement in the usual way.

In another embodiment, with a sharp towel clip or other device to reapproximate the medial knee retinaculum, the sharp point of the computer tracked point probe is driven manually 1-2 mm into the bone of the anterior patella in roughly the center of the patella. With the computer tracking the position of the femur and the position of the point probe, the knee is flexed and/or extended. The computer samples data points at a rapid rate to generate a line in space that represents the movement of the tip of the probe (the movement of the patella, since the tip of the probe is embedded in the patella) in relation to the femur. A reference line is thus obtained on the distal end of the femur for making bone cuts as described in the previous embodiment.

In an alternative embodiment, an optical or laser or electromagnetic or mechanical tracker is attached to the anterior surface of the patella after exposure of the profile of the femur and tibia. The knee is then flexed and/or extended and the movement of the patella recorded as before. The computer is programmed to project this line in space onto the transverse plane of the femur. This line represents the desired anterior/posterior axis of the femoral component. Bone cuts on the distal femur are then made using this line as a guide to rotational positioning of the femoral component.

Similarly, the alignment line is found for the tibial component by projecting the movement of the patella on to the proximal surface of the tibia, as shown in FIG. 7. The resected bone surface on the proximal tibia can then be prepared such that the anterior-posterior axis of the tibial component is aligned with the projected line 720.

In a preferred embodiment of the invention, the tracking technique is intended to be used with a computer-assisted surgery system. After the knee has been exposed and trackers have been placed in the femur and the tibia, but before any bone cuts are made, a tracking device is attached to the patella either on its underside or on the upper surface. The knee is then flexed and/or extended. Positional data is gathered at 60 Hz using a data acquisition system and stored in the computer. As described previously, the data is smoothed and fitted to a curve and stored in the computer. The projected axis is then used as a reference during the surgery for making the bone cuts prior to affixing the prostheses.

It has been shown that the present method results in errors of rotational alignment either equal to or less than the best methods that are currently available, as shown in Tables 1 and 2. Table 1 shows the experimentally determined errors in a study including 12 surgeons for the present technique when compared to various prior art techniques of referencing the alignment axis. The consolidated mean errors for the various techniques are given in Table 2. By “Clinical Axis” we mean the axis that is lateral prominence to medial prominence. By “Surgical Axis” we mean the lateral prominence to medial sulcus.

It is observed that the error and variation in error for the patella technique is comparable to existing techniques in some cases, and better in others. In the case of the Digitized Epicondyles approach, the error is smaller (i.e., closer to zero error with respect to the clinical axis, as shown in Table 2); however, the large errors of internal rotation make this method unsuitable, since internal rotational errors have serious consequences including maltracking of the patella. The error with reference to the surgical axis for the Whiteside's line technique (−2.3°) is slightly less than that of the patella technique (−2.6°). However, the standard deviation of ±8.8° is more than that of patella technique (±7.7°). Further, referencing using the Whiteside's line technique may be difficult when there is damage to the end of the femur because of disease or in revision arthroplasty. It is clear from Table 2 that the patella technique resulted in one of the lowest internal rotation errors, suggesting the promise of this technique.

TABLE 1
Errors in Determining Surgical Epicondylar Axis by Surgeon for the Patellar
Technique with Reference to Prior Art Techniques

| | | | | | | Surgeon's |
|---|---|---|---|---|---|---|
| | Digitized | Whiteside's | Posterior | Patella | | Mean |
| | Epicondyles | Line | Condyles | Tracking | Screw Axis | Alignment |
| Surgeon 1 | 0.9° ± 8.3° | −2.7° ± 7.0° | −0.9° ± 8.8° | −2.5° ± 6.6° | 11.4° ± 4.2° | 1.2° ± 8.6° |
| Surgeon 2 | 2.0° ± 7.1° | −0.5° ± 4.6° | −6.1° ± 9.4° | −6.5° ± 15.6° | 10.4° ± 4.5° | −0.2° ± 10.8° |
| Surgeon 3 | 7.5° ± 7.3° | −1.7° ± 6.5° | −3.8° ± 7.3° | −1.8° ± 6.0° | 7.8° ± 7.7° | 1.6° ± 8.3° |
| Surgeon 4 | 6.4° ± 8.0° | 2.2° ± 5.6° | 0.1° ± 9.1° | −2.6° ± 10.5° | 10.6° ± 6.0° | 3.4° ± 9.0° |
| Surgeon 5 | 5.1° ± 8.3° | −2.9° ± 19.9° | −1.8° ± 8.9° | −2.2° ± 6.2° | 10.7° ± 4.4° | 1.8° ± 11.6° |
| Surgeon 6 | 8.8° ± 4.5° | −2.1° ± 6.3° | −1.8° ± 11.5° | −4.4° ± 5.2° | 12.0° ± 4.1° | 2.5° ± 9.4° |
| Surgeon 7 | 4.7° ± 4.0° | 0.8° ± 4.3° | −9.4° ± 12.2° | −2.9° ± 8.1° | 9.6° ± 8.1° | 0.5° ± 10.0° |
| Surgeon 8 | 6.6° ± 6.8° | −5.6° ± 6.4° | −1.5° ± 13.9° | −2.1° ± 6.2° | 9.8° ± 5.0° | 1.4° ± 9.9° |
| Surgeon 9 | 1.9° ± 7.0° | −3.2° ± 10.7° | −4.2° ± 6.0° | −0.6° ± 5.5° | 10.4° ± 5.8° | 0.9° ± 8.7° |
| Surgeon 10 | 7.0° ± 5.2° | −6.9° ± 11.6° | −5.2° ± 6.9° | −3.3° ± 7.4° | 9.1° ± 8.5° | 0.1° ± 10.3° |
| Surgeon 11 | 3.1° ± 8.4° | −1.9° ± 7.3° | −3.5° ± 7.7° | −1.1° ± 6.7° | 11.6° ± 4.5° | 1.6° ± 8.7° |
| Surgeon 12 | 10.7° ± 4.5° | −3.0° ± 6.6° | 8.3° ± 18.6° | −1.6° ± 5.1° | 12.1° ± 5.0° | 5.3° ± 11.1° |
| Technique | 5.4° ± 7.1° | −2.3° ± 8.8° | −2.5° ± 10.9° | −2.6° ± 7.7° | 10.5° ± 5.7° | |
| Mean | | | | | | |

TABLE 2
Mean Errors With Reference to Clinical and Surgical Axes for the Patellar
Technique Compared to Prior Art Techniques

| | Digitized | Whiteside's | Posterior | Patella | |
|---|---|---|---|---|---|
| | Epicondyles | Line | Condyles | Tracking | Screw Axis |
| Error w.r.t. | −0.3° ± 7.9° | −5.3° ± 9.1° | −6.6° ± 9.9° | −6.5° ± 7.8° | 3.7° ± 9.1° |
| clinical axis | | | | | |
| Error w.r.t. | 5.4° ± 7.1° | −2.3° ± 8.8° | −2.5° ± 10.9° | −2.6° ± 7.7° | 10.5° ± 5.7° |
| surgical axis | | | | | |

The present method enables accurate angular positioning of the alignment axes of the bones at the knee joint using a method that is independent of the shape and condition of the femur and the tibia. It is therefore shown to be as good as or better than other currently used methods of computer-assisted navigation. It is likely that combining techniques that are based on physically tracking the relative positions of the bones by attaching markers, such as the patella tracking technique, and imaging the profile of the bones using an imaging technique, such as X-ray fluoroscopy, CT scanning or otherwise, would yield better outcomes. In general, the patellar techniques presented herein may be combined with other kinematic or anatomic techniques to compute a (combined) rotational alignment axis, for example by averaging two axes obtained according to two different techniques to obtain a third combined axis to be used in an actual surgical procedure, thereby providing improved accuracy in rotational alignment of femoral components. Advantageously, while rotational alignment outliers may still pose problems, they are reduced when such combination techniques are used.

In one such embodiment, computer 540 computes a first alignment axis of the femoral component according to the patellar technique described above. Additionally, the computer 540 stores (or computes) a second alignment axis according to a second technique. The second alignment axis may be obtained according to an anatomical technique such as “Digitized Epicondyles,” “Whiteside,” or “Posterior Condyles.” In one embodiment, the computer 540 may simply obtain the second alignment axis from another computing system used to compute or store a digital representation of the second axis. Alternatively, computer 540 may itself compute the second axis, in which case the computer 540 may obtain input data necessary for such computation via an optically-tracked stylus or other accessories used by surgeons to identify anatomical landmarks used in the determination of the second axis. Alternatively, the second alignment axis may be obtained according to a kinematic technique such as “Screw Axis,” in which case the computer 540 (or another kinematic navigation system in communication with computer 540) records the position and orientation of optical trackers attached to the femur, tibia, patella, or other sites used to kinematically determine the second axis.

Once the first and second alignment axes are available to the computer 540, the computer 540 computes a third alignment axis by combining the first and second axes. The third axis may be generated as a straight average of the first and second axes, or it may be generated as a weighted average or other numerical or geometric combination of the first and second axes. This combined third alignment axis is then used to perform the surgery. As should be obvious to one of ordinary skill in the art, the technique of combining alignment axes can be extended to combining more than two alignment axes.

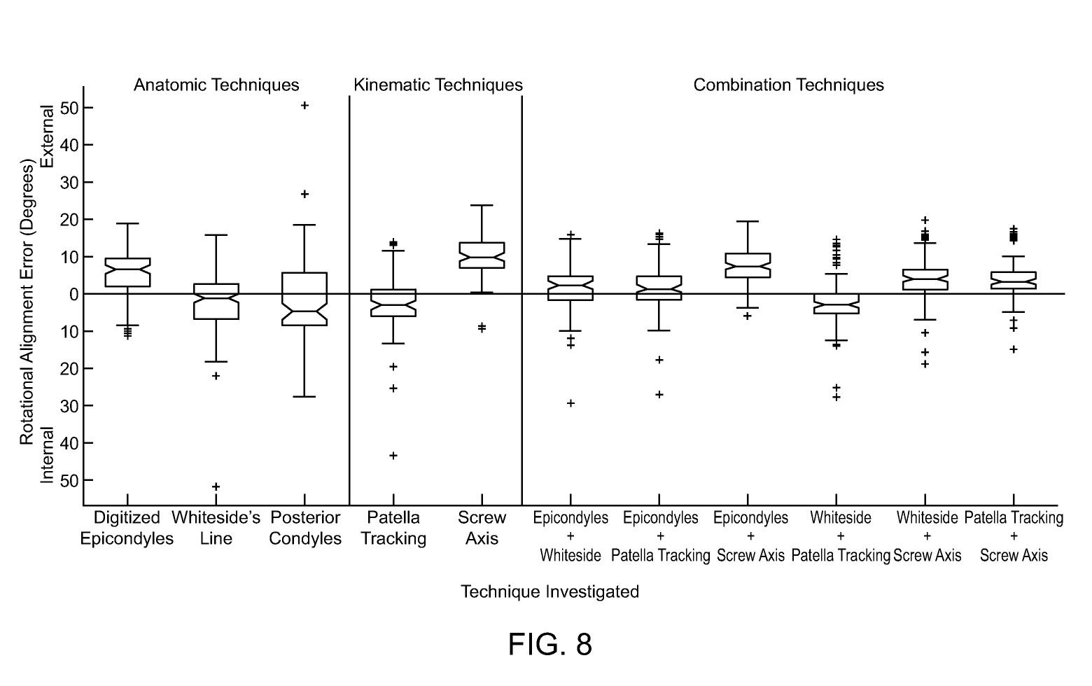

It has been shown that the combination techniques are precise and more accurate than single methods, as shown in Table 3 and FIG. 8. In a study including 12 surgeons, 58% (375 of 648) of the anatomic axes were rotated less than 5° from a reference axis. The “Epicondyles+Whiteside”, “Epicondyles+Patella Tracking”, “Whiteside+Patella Tracking” and “Patella Tracking+Screw Axis” techniques were the most accurate techniques in the study.

TABLE 3
Error in Femoral Rotational Alignment with Respect to
Surgical Epicondylar Axis

| Technique Name | Alignment Error (mean ± std deviation) |
|---|---|
| Digitized Epicondyles | 5.4° ± 7.1° |
| Whiteside's Line | −2.3° ± 8.8° |
| Posterior Condyles | −2.5° ± 10.9° |
| Patella Tracking | −2.6° ± 7.7° |
| Screw Axis | 10.5° ± 5.7° |
| Epicondyles + Whiteside | 1.5° ± 6.6° |
| Epicondyles + Patella Tracking | 1.4° ± 6.4° |
| Epicondyles + Screw Axis | 7.9° ± 5.3° |
| Whiteside + Patella Tracking | −2.5° ± 6.5° |
| Whiteside + Screw Axis | 4.1° ± 5.9° |
| Patella Tracking + Screw Axis | 3.9° ± 5.4° |

Positive values represent external rotation

Although the above examples have been illustrated with respect to the knee joint, it would be clear to those familiar with the art that the method is applicable, in general, for establishing the alignment of any two bones at a joint using the motion of a third related bone. For example, the motion of the ulna with respect to the humerus at the elbow can be used to align components for a total shoulder arthroplasty, the motion of the femur with respect to the tibia at the knee can be used to align the components of a total ankle arthroplasty or the motion of the tibia with respect to the femur can be used to align the joint for total hip replacement.

What is claimed is:

1. A method of determining the rotational alignment axis of bone joints involving a first and a second bone, comprising:
tracking the position of a third bone with reference to the first or the second bone;
obtaining a trajectory of the third bone, wherein the trajectory is generated by the movement of the bone joint; and
calculating an axis of alignment of the first or second bone at the joint using the relative movement of the third bone with reference to either the first or the second bone.

2. The method of claim 1, wherein the tracking comprises using imaging to track the third bone with reference to the first or the second bone.

3. The method of claim 2, wherein the imaging comprises X-ray imaging, computed tomography imaging, fluoroscopic imaging, magnetic resonance imaging, or ultrasound imaging.

4. The method of claim 1, wherein the tracking comprises using a tracker attached to the third bone.

5. The method of claim 4, wherein the tracker is an optical sensor.

6. The method of claim 4, wherein the tracker is an electromagnetic sensor.

7. The method of claim 4, wherein the tracker is a mechanical sensor.

8. The method of claim 1, wherein the first bone is the femur, the second bone is the tibia and the third bone is the patella.

9. The method of claim 1, wherein the first bone is the femur and the second bone is the pelvis and the third bone is the tibia.

10. The method of claim 1 wherein the first bone is the femur, the second bone is the tibia and the third bone is the talus.

11. A method of determining the rotational alignment axis of the femur at the knee joint, comprising:
tracking the position of the patella within the femoral groove as the joint is flexed or extended, using a tracker attached to the patella;
obtaining the trajectory of the patella with reference to the femoral groove as the knee is flexed or extended; and
calculating the axis of rotational alignment of the femur at the knee joint from data indicating a curve of motion of the patella by fitting an appropriate function to the data.

12. The method of claim 11, further comprising:
projecting the fitted function on to a cross-sectional plane perpendicular to the anatomical axis of the femur.

13. The method of claim 11 where the data is projected on to a plane perpendicular to the mechanical axis of the femur.

14. The method of claim 11, wherein the fitting function is a spline function.

15. The method of claim 11, wherein the fitting function is a polynomial function.

16. The method of claim 11, wherein the data is filtered using a Fourier filtering scheme.

17. A method of determining the rotational alignment axis of the tibia at the knee joint, comprising:
tracking the position of the patella with reference to the tibia as the joint is flexed or extended, using a tracker attached to the patella;
obtaining the trajectory of the patella with reference to proximal end of the tibia as the knee is flexed or extended; and
calculating the axis of rotational alignment of the femur at the knee joint from data indicating a curve of motion of the patella by fitting an appropriate function to the data.

18. The method of claim 17, further comprising:
projecting the fitted function on to the cross-sectional plane perpendicular to the anatomic axis of the tibia.

19. The method of claim 17, wherein the fitting function is a spline function.

20. The method of claim 17, wherein the fitting function is a polynomial function.

21. The method of claim 17, wherein the data is filtered using a Fourier filtering scheme.

22. A computerized bone tracker system comprising:
a bone tracker adapted to be attached to a bone, wherein the bone is connected to a first bone and a second bone;
a computer system capable of tracking the movement of the bone tracker, wherein the computer system comprises a sensor, a data processing unit and a display unit and the computer system obtains a trajectory of the bone tracker as the bone tracker tracks the movement of the bone it is attached to and calculates an axis of rotational alignment of the first or second bone at the joint using the relative movement of the bone with reference to either the first or the second bone.

23. The system of claim 22, wherein the bone is patella, the first bone is tibia and the second bone is femur.

24. The system of claim 23, wherein the sensor is an optical sensor.

25. The system of claim 23, wherein the sensor is an electromagnetic sensor.

26. The system of claim 23, wherein the sensor is a mechanical sensor.

27. A computer-implemented method for generating a rotational alignment axis of bone joints involving a first and a second bone, comprising:
computing a first rotational alignment axis according to a first arthroplasty technique;
obtaining a second rotational alignment axis computed according to a second arthroplasty technique; and
combining the first and second axes to generate a third rotational alignment axis of the first or second bone at the joint.

28. The method of claim 27, wherein the computing the first axis comprises:
obtaining a trajectory of a third bone with reference to the first or the second bone,
wherein the trajectory is generated by a tracker attached to the third bone during normal movement of the bone joint; and
computing an axis of alignment of the first or second bone at the joint using the relative movement of the third bone with reference to either the first or the second bone.

29. The method of claim 27, wherein the first bone is the femur, the second bone is the tibia and the third bone is the patella.

30. The method of claim 27, wherein the combining comprises computing an average of the first and second rotational alignment axes.

31. The method of claim 30, wherein the average is a weighted average.

32. The method of claim 27, wherein the obtaining comprises computing the second rotational alignment axis according to the second arthroplasty technique.

33. The method of claim 27, wherein the obtaining comprises communicating with a kinematic navigation system to receive the second rotational alignment axis.
